(19) United States
(12) Patent Application Publication
(10) Pub. No.: US 2025/0017655 A1
(43) Pub. Date: Jan. 16, 2025

(54) METHOD OF DESIGNING PATIENT-SPECIFIC IMPLANT AND GUIDANCE, AND PROGRAM AND APPARATUS THEREFOR

(71) Applicant: SeeAnn Solution Co., Ltd., Incheon (KR)

(72) Inventors: Anna SEO, Incheon (KR); Youngjin JEONG, Incheon (KR); Hakjong NOH, Incheon (KR); Chunghwan BAEK, Seoul (KR)

(73) Assignee: SeeAnn Solution Co., Ltd., Incheon (KR)

(21) Appl. No.: 18/416,537

(22) Filed: Jan. 18, 2024

(30) Foreign Application Priority Data
Jul. 11, 2023 (KR) 10-2023-0089763

(51) Int. Cl. A61B 34/10

(52) U.S. Cl. CPC A61B 34/10; A61B 2034/104; A61B 2034/105; A61B 2034/107; A61B 2034/108

(57) ABSTRACT

Provided is a method of designing an implant and a guidance specific to a patient, and a program and apparatus therefor. The method includes: specifying a first skeletal image of a skeleton of a patient in which an affected region to which an implant is to be implanted is located; specifying a second skeletal image of another region skeleton including the implant; calculating a number of at least one implant matching the first skeletal image; cutting the second skeletal image according to the calculated number of implants such that the at least one implant matches the first skeletal image, and displaying the cut second skeletal image; and displaying the cut at least one implant to be overlaid on the first skeletal image.

### CROSS-REFERENCE TO RELATED APPLICATION

This application claims priority to and the benefit of Korean Patent Application No. 10-2023-0089763, filed on Jul. 11, 2023, the disclosure of which is incorporated herein by reference in its entirety.

### BACKGROUND

### 1. Field of the Invention

The disclosed technology relates to designing an implant to be implanted into a patient's cut area and designing a guidance for cutting an cut part or a corresponding implant.

### 2. Discussion of Related Art

With the advancement of medical technology, there have been ongoing efforts to replace damaged human body tissues with tissues from other parts of the body.

For example, when oral cancer occurs in a lower jaw (lower jaw skeleton) area of a patient and thus cutting the bone in the corresponding site is considered the most obvious treatment, it is common to cut the bone in the corresponding site, cut a suitable skeleton portion from a bone (referred to as the fibula) on the side of the shin bone, and implant the skeleton portion.

Since such surgeries have high risk and even minor errors may cause irreversible damage to the patient, there is a need to design a to-be cut portion and a to-be implanted portion in advance in a virtual three-dimensional space, and design a guidance that helps to reduce errors when cutting actual bones.

### SUMMARY OF THE INVENTION

The present invention is directed to simulating an implantation process by specifying a skeleton of a to-be cut site and a skeleton of a to-be implanted site in a virtual three-dimensional space.

The present invention is directed to designing a guidance in a virtual three-dimensional space to fix a to-be cut site to reduce errors when cutting the actual skeleton.

The present invention is directed to designing a cut portion into a plurality of cut regions such that the cut portion is appropriately implanted into the skeleton of a site subject to implantation.

The technical challenges that this embodiment aims to achieve are not limited to the technical challenges described above, and other technical challenges may be inferred from the following embodiments.

According to an aspect of the present invention, there is provided a design method, which is a method performed by an apparatus for designing an implant and a guidance specific to a patent, the design method including: specifying a first skeletal image of a skeleton of a patient in which an affected region to which an implant is to be implanted is located; specifying a second skeletal image of another region skeleton including the implant; calculating a number of at least one implant matching the first skeletal image; cutting the second skeletal image according to the calculated number of implants such that the at least one implant matches the first skeletal image, and displaying the cut second skeletal image; and displaying the cut at least one implant to be overlaid on the first skeletal image.

The specifying of the first skeletal image may include: generating one or more planes around the affected region; and specifying, as the first skeletal image, a specific closed area corresponding to a cutting target area among one or more closed areas defined in the first skeletal image by the one or more planes.

The calculating of the number of the at least one implants matching the first skeletal image may include specifying, by a user, coordinates of n vertices inside or outside the first skeletal image.

The cutting of the second skeletal image according to the calculated number of implants such that the at least one implant material matches the first skeletal image, and the displaying of the cut second skeletal image may include: generating two cutting planes to remove the specific closed area; designating first lines passing through the two cutting planes; generating cutting planes n−1 from the coordinates of the vertices; aligning the first lines into a straight line; generating an oriented bounding box (OBB) for the other area skeleton including the second bone image, and connecting center points of two minimum planes of the OBB to designate a second line; matching the first line with the second line; and cutting the other region skeleton based on the first line.

The design method may further include: generating cutting guide pieces based on the OBB for the second skeletal image; generating a bridge structure connecting the cutting guide pieces; and generating a guidance by combining the cutting guide pieces and the bridge structure.

The displaying of the cut at least one implant to be overlaid on the first skeletal image may include, when a user arbitrarily moves the coordinates of the vertex, performing the cutting of the second skeletal image according to the calculated number of implants such that the at least one implant matches the first skeletal image and the displaying of the cut second skeletal image again.

The displaying of the cut at least one implant to be overlaid on the first skeletal image may include allowing a user to arbitrarily perform coordinate change, turning, or spinning on the cut at least one implant displayed to be overlaid on the first skeletal image.

The displaying of the cut at least one implant to be overlaid on the first skeletal image may include displaying the at least one implant to be overlaid on the first skeletal image while displaying the at least one implant with respect to the other region skeleton including the second skeletal image.

According to an aspect of the present invention, there is provided an apparatus for designing an implant and a guidance specific to a patent, the apparatus including: an input/output interface, a memory in which an instruction is stored, and a processor, wherein the processor is configured to, in connection with the memory, specify a first skeletal image of a skeleton of a patient in which an affected region to which an implant is to be implanted is located; specify a second skeletal image of another region skeleton including the implant; calculate a number of at least one implant matching the first skeletal image; cut the second skeletal image according to the calculated number of implants such that the at least one implant matches the first skeletal image, and display the cut second skeletal image; and display the cut at least one implant to be overlaid on the first skeletal image.

According to an aspect of the present invention, there is provided a computer readable recording medium on which a program for performing a method performed by an apparatus for designing an implant and a guidance specific to a patient according to a disclosed embodiment is recorded.

Specific details of other embodiments are included in the detailed description and drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a flowchart for describing a design method according to an embodiment;

FIG. 2 illustrating a cutting target skeletal image and an implantation target skeletal image according to an embodiment;

FIGS. 3A to 3E illustrate a skeletal image dividing process of dividing a first implantation target skeletal image, which is an implantation target skeletal image before exclusion of an implantation target area, into a plurality of implantation target areas according to an embodiment;

FIGS. 4A to 4H illustrate a user modeling process of modeling a plurality of cutting target skeletal images suitable for a plurality of implantation target skeletal images, according to an embodiment of the present invention;

FIGS. 5A to 5C illustrates a process of designing a first guidance according to one embodiment;

FIGS. 6A to 6B illustrate a plurality of cutting target skeletal images, which are designed by the first guidance, overlaid on a second implantation target skeletal image, according to an embodiment;

FIG. 7A is a perspective view of the first guidance according to an embodiment, and FIG. 7B is a view of the first guidance overlaid on the cutting target skeletal image, according to an embodiment; and

FIG. 8 is a block diagram illustrating the structure of a design apparatus according to an embodiment.

### DETAILED DESCRIPTION OF EXEMPLARY EMBODIMENTS

Although terms used herein are selected from among general terms that are currently and widely used in consideration of functions in the exemplary embodiments, these may be changed according to intentions or customs of those skilled in the art or the advent of new technology. However, when a specified term is defined and used in an arbitrary sense, a meaning of the term will be described in the specification in detail. Accordingly, the terms used herein are not to be defined as simple names of the components but should be defined based on the actual meaning of the terms and the whole context throughout the present specification.

Throughout the specification, the term “comprises” or “includes” and/or “comprising” or “including” means that one or more other components may not be further excluded unless context dictates otherwise. In the specification, the term “part” or “module” refers to a unit for processing at least one function or operation that may be implemented in hardware, software, or a combination thereof.

As used herein, expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

In the following description, terms such as “transmission,” “communication,” “sending,” “reception,” and other similar meanings of signals, messages, or information are not only meant to directly convey signals, messages, or information from one component to another. It also includes passing through other components.

In particular, “transmitting” or “sending” a signal or information to a component indicates the final destination of the signal or information and does not mean a direct destination. The same is true for the “reception” of a signal or information. In addition, in this specification, that two or more pieces of data or information are “related” means that if one data (or information) is obtained, at least a portion of the other data (or information) may be obtained based thereon.

It should be understood that, although the terms “first,” “second,” “third,” etc. may be used herein to describe various elements, these elements are not limited by these terms. These terms are only used for distinguishing one element from another.

For example, a first element could be termed a second element or a third element without departing from the scope of the present invention.

Although embodiments of the present disclosure will be described in detail with reference to the accompanying drawings in order to enable those skilled in the art to easily practice the disclosure, the present disclosure may be embodied in many different forms and should not be construed as being limited to the embodiments set forth herein.

Hereinafter, embodiments of the present invention will be described in detail with reference to the attached drawings.

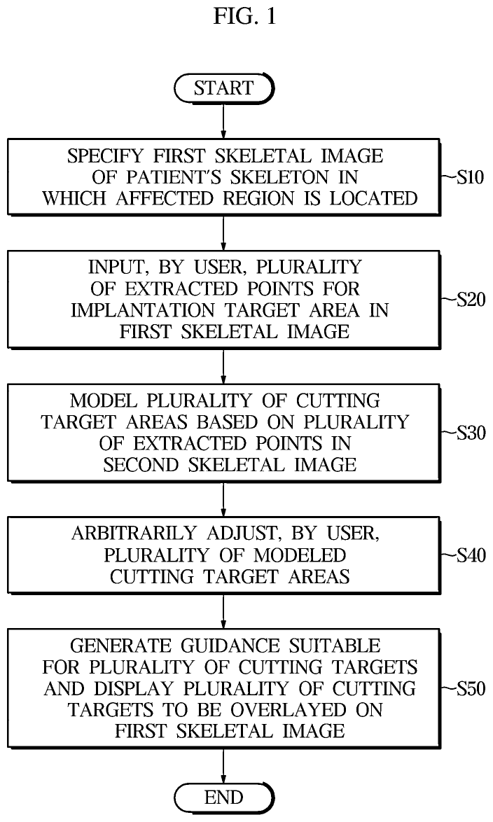

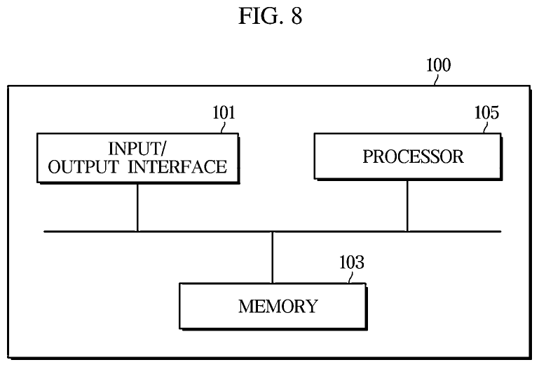

FIG. 1 is a flowchart for describing a design method according to an embodiment. The method shown in FIG. 1 may be performed by an electronic apparatus 100 described with reference to FIG. 8. In FIG. 1, the method of designing an implant and a guidance is described as being divided into a plurality of operations, but at least some of the operations may be performed in a reverse order, performed in combination with other operations, omitted, further divided into a larger number of sub-operations, or combined into a smaller of operations. In addition, some operations described herein may be added.

An apparatus for designing an implant and a guidance specific to a patient (hereinafter referred to as a “design apparatus”) specifies a first skeletal image of a patient's skeleton in which an affected region to which an implant is to be implanted is located (S10). In this case, as an example, the “patient's skeleton” may include a lower jaw (a lower jaw bone) of a facial area in which an affected region is located.

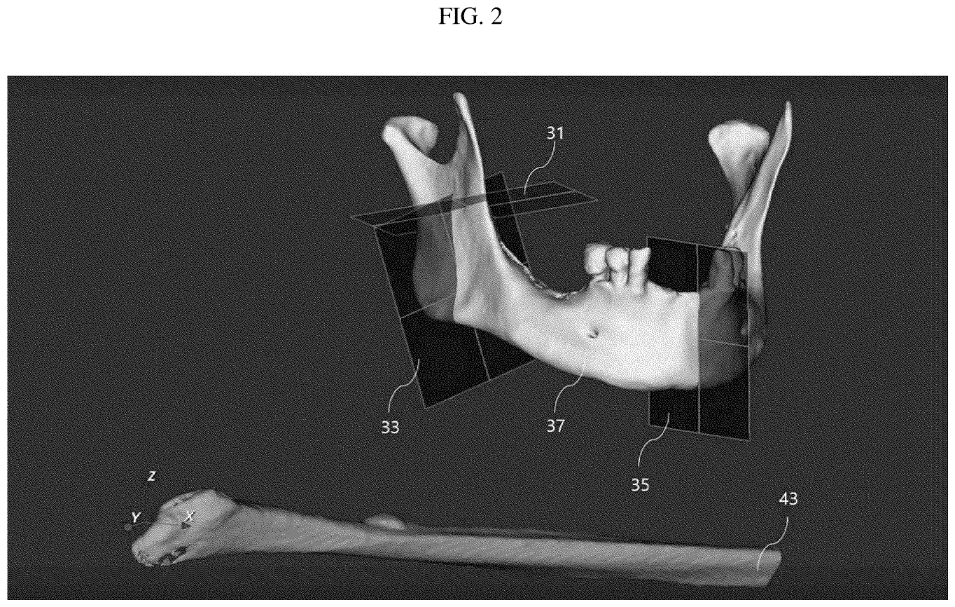

In this regard, a process of specifying the first skeletal image is omitted, but referring to FIG. 2, in the embodiment, the design apparatus 100 may generate one or more planes 31, 33, and 35 around the affected region, specify a specific closed area 37, which corresponds to a cutting target area, among one or more closed areas defined by the one or more planes 31, 33, and 35 in the first skeletal image as the first skeletal image, and the specific closed area 37 may be defined as an implantation target skeletal image according to an embodiment.

In addition, when the design apparatus models a second skeletal image of another region skeleton including an implant. In this case, the “the other region skeleton” is a region to be cut and may be defined as a cutting target image area 43. As an example, the fibula may be used as the region to be cut, but the disclosure is not limited thereto. That is, as another example, a human skeleton that may be used as a region to be cut may include the pelvic bone, hip bone, and the like.

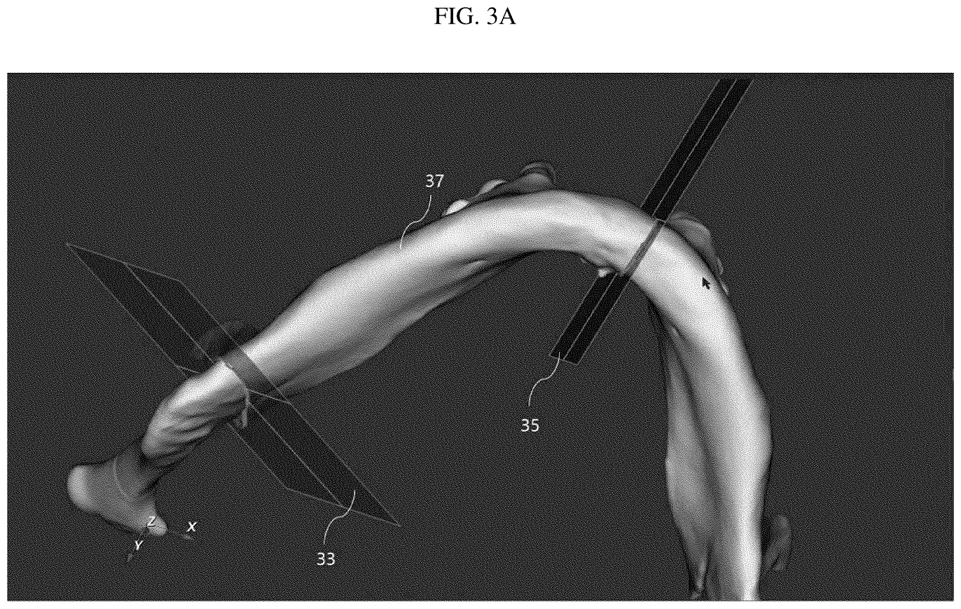

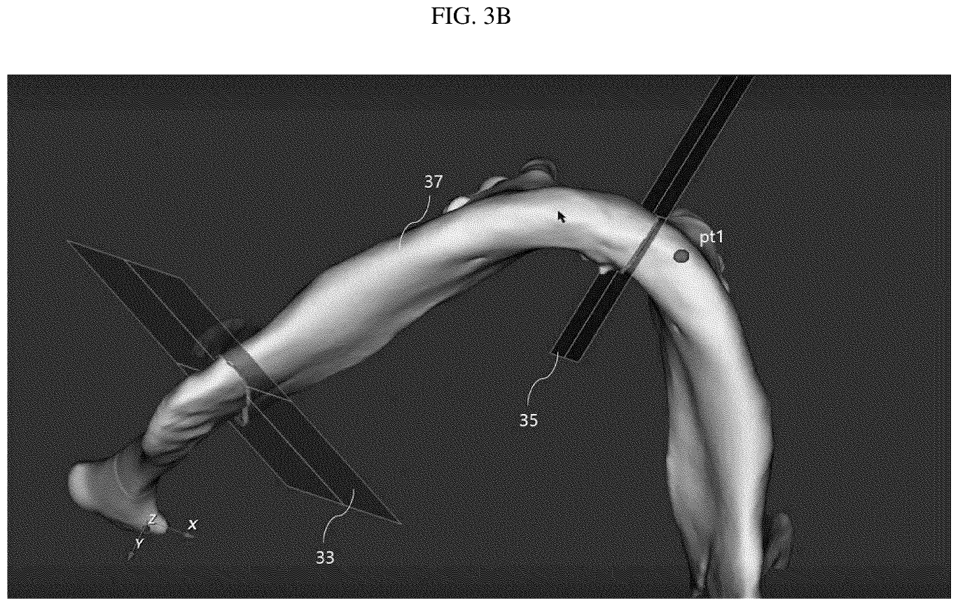

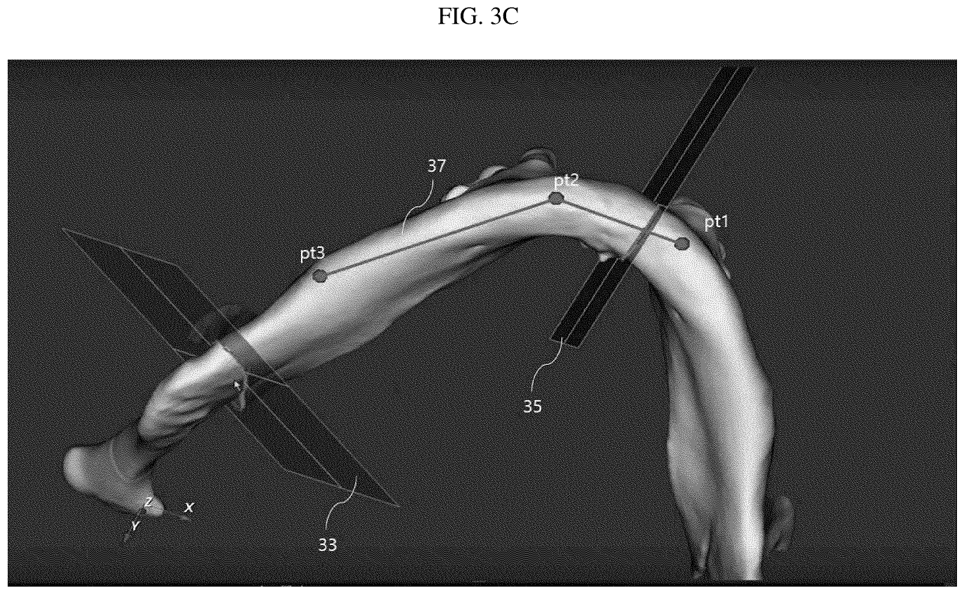

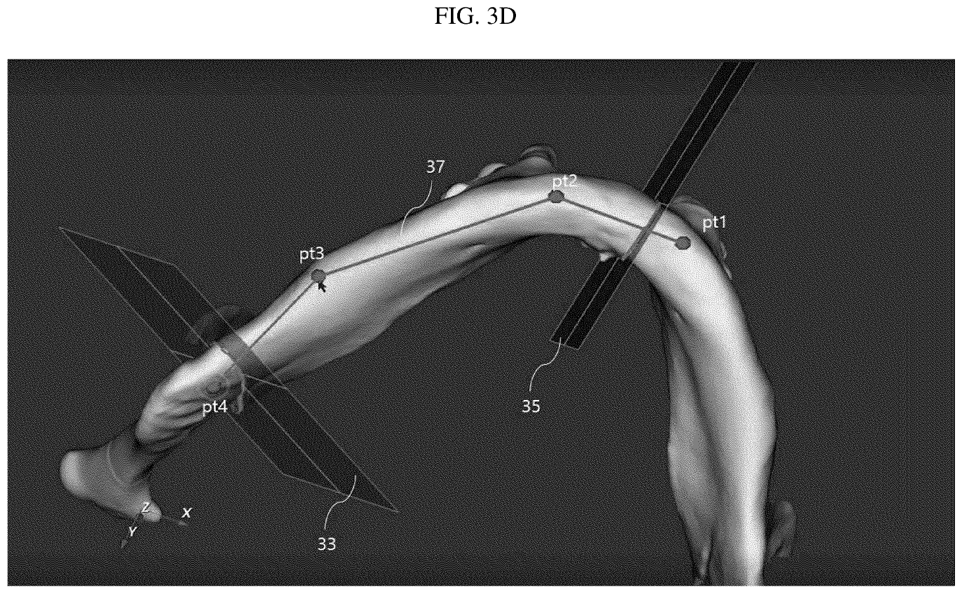

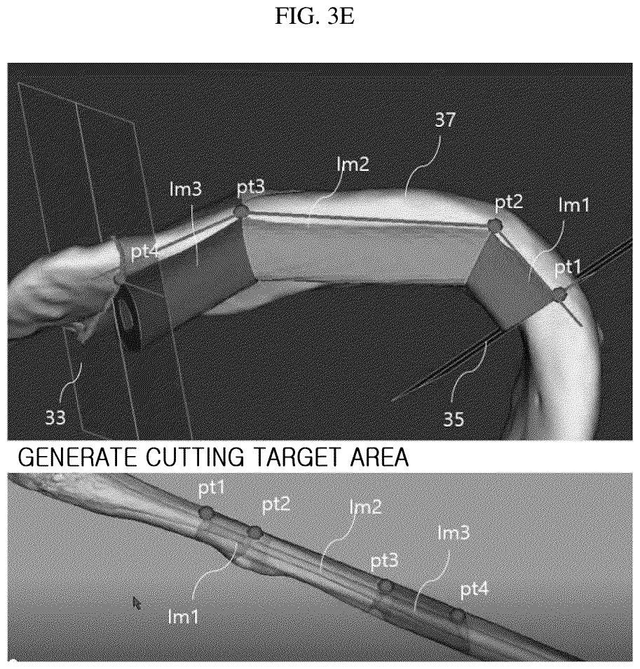

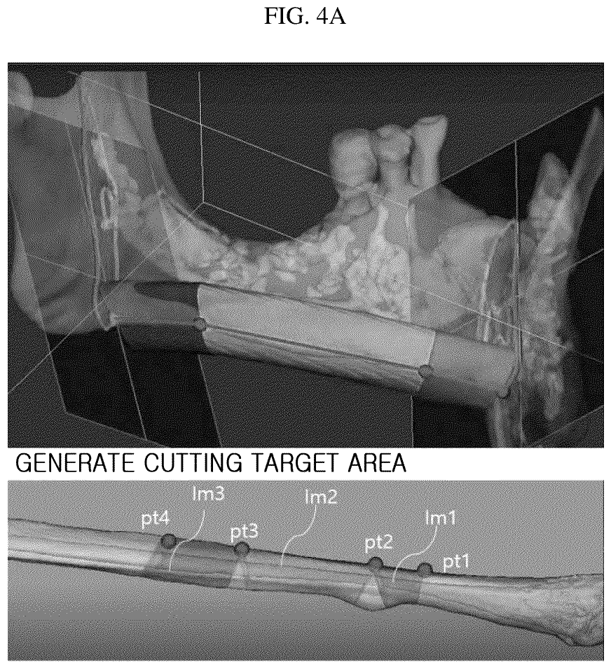

The design apparatus receives input of a plurality of vertices for the implantation target area in the first skeletal image (S20). In operation S20 and FIGS. 3A to 3D described below, a plurality of vertices are described as being input from the user, but the number and positions of a plurality of vertices may be arbitrarily generated based on the cutting target image area and thickness and shape information of the bone included in the implantation target image area according to data learned by a processor in the design apparatus 100.

FIGS. 3A to 3E illustrate a skeletal image dividing process of dividing a first implantation target skeletal image, which is an implantation target skeletal image before exclusion of an implantation target area, into a plurality of implantation target areas according to an embodiment.

FIGS. 3A to 3E illustrate 3D images of the first skeletal image shown in FIG. 2, which are viewed from below.

As in FIG. 2, in FIGS. 3A to 3E, a specific closed area 37 is determined by one or more planes 31, 33, and 35, and the specific closed area is defined as an implantation target image area.

In some cases, the implantation target image area may have a curved bone shape. When an implant object is a straight cut object, such as the fibula, it is important to divide the object of implant into a plurality of implant objects and implant the plurality of implant objects.

That is, when the fibula is implanted between the first plane 35 and the second plane 33 of the specific closed area without being divided into a plurality of cutting target areas, the cutting target area implanted to the specific closed area may generate an artificial external appearance of heterogeneity, unlike the normal lower jaw. Therefore, in order to minimize external heterogeneity and perform implantation with a skeleton similar to a normal lower jaw, the user may input a plurality of vertices for the implantation target area in the first skeletal image (S20 in FIG. 1).

Referring to FIG. 3B, a first vertex pt1 is extracted from the right side of the first plane 35 for dividing the cutting target area, and referring to FIG. 3C, a second vertex pt2 and a third vertex pt3 are extracted from the left side of the first plane 35 for dividing the cutting target area.

However, it is also possible to designate the first vertex pt1 on the first plane 35.

Also, referring to FIG. 3D, a fourth vertex pt4 is extracted on the second plane 33 for dividing the cutting target area.

In FIGS. 3A to 3D, four vertices are input to generate a plurality of implants, but the user may arbitrarily determine the number of the vertices, or the design apparatus 100 may appropriately adjust the number of vertices according to learning.

Here, adjusting the number of vertices according to learning may be determined according to the thickness of the bone, the degree of curvature of the bone, the length of the bone, and the like.

Therefore, referring to FIG. 3E, the design apparatus 100 generates three implants Im1, Im2, and Im3 between the four vertices and the two planes (the first plane 35 and the second plane 33) based on the four vertices extracted as in FIGS. 3A to 3D. The design apparatus 100 according to the present invention may receive a user's designation for each vertex through an input/output interface 101 and display position information about the corresponding vertex on an implantation target area image such that the user may check the position of the corresponding vertex.

In addition, as shown in FIG. 3E, the input/output interface 101 may output information about the three generated implants while simultaneously representing each vertex and the first to third implants to be distinguished on the cutting target area.

Accordingly, the user may simultaneously check the implantation target image area and the cutting target image area correlated with the extracted vertices, thereby facilitating checking whether the vertices have been accurately extracted, and modifying the vertices.

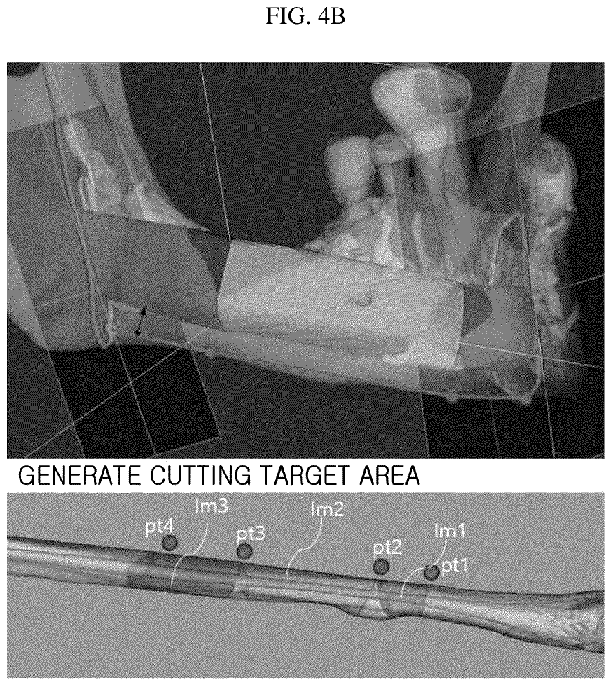

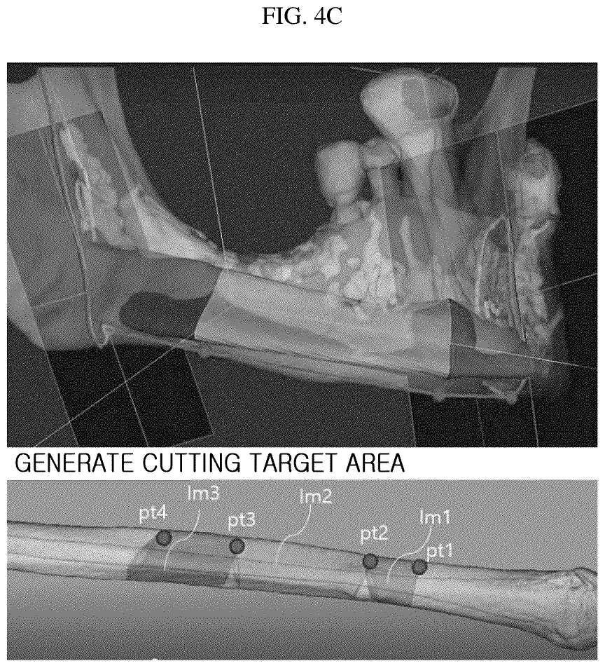

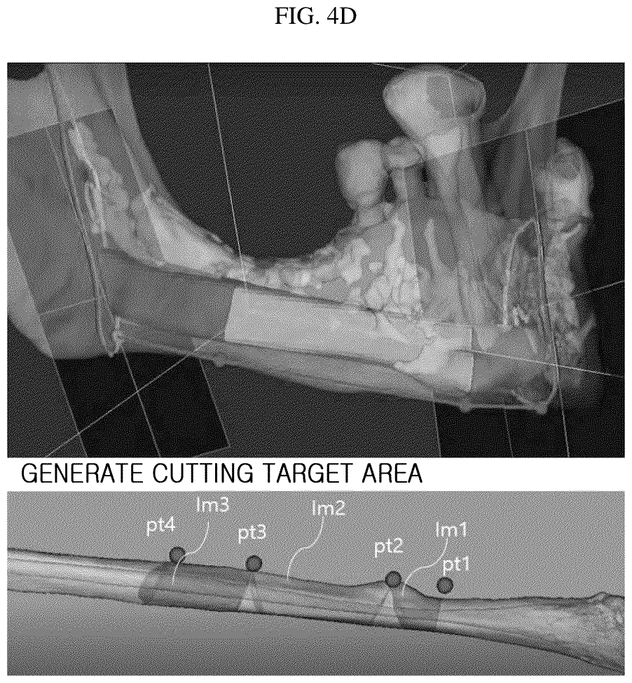

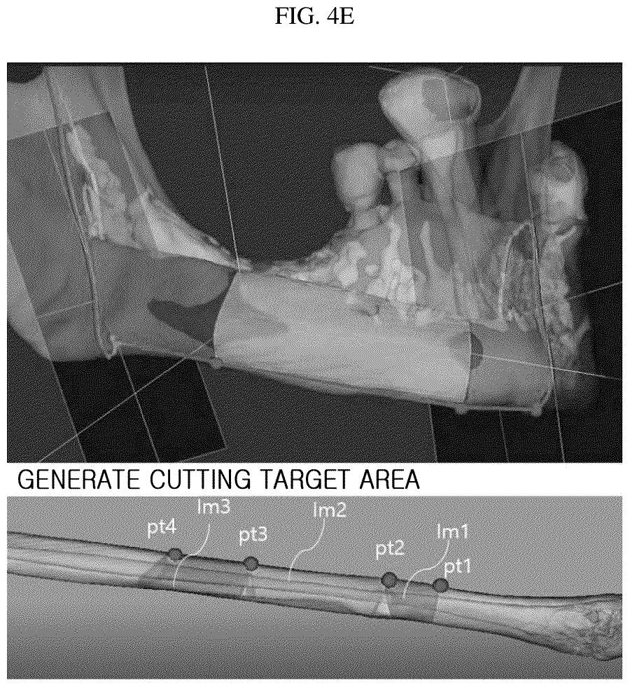

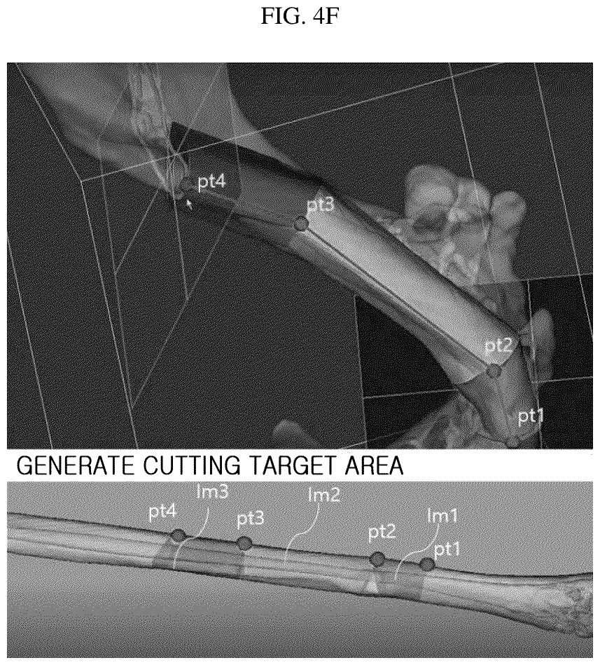

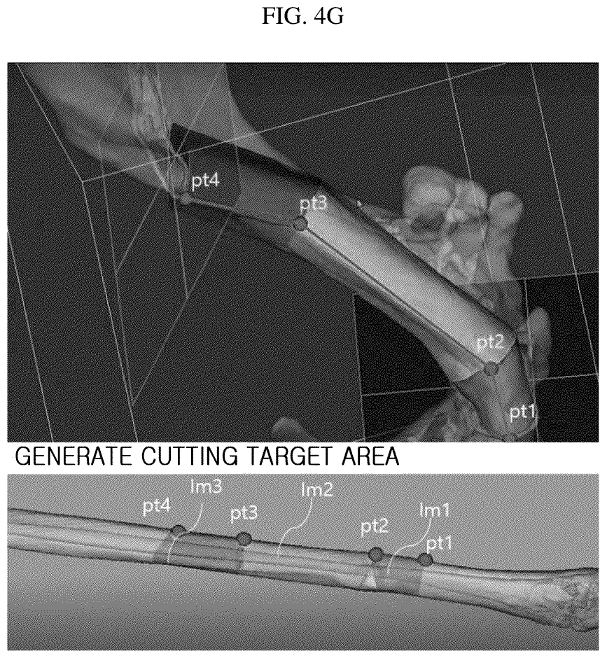

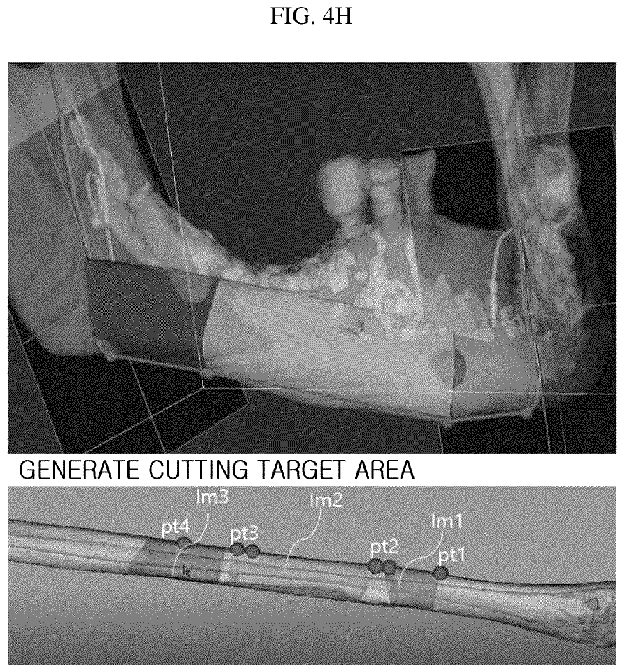

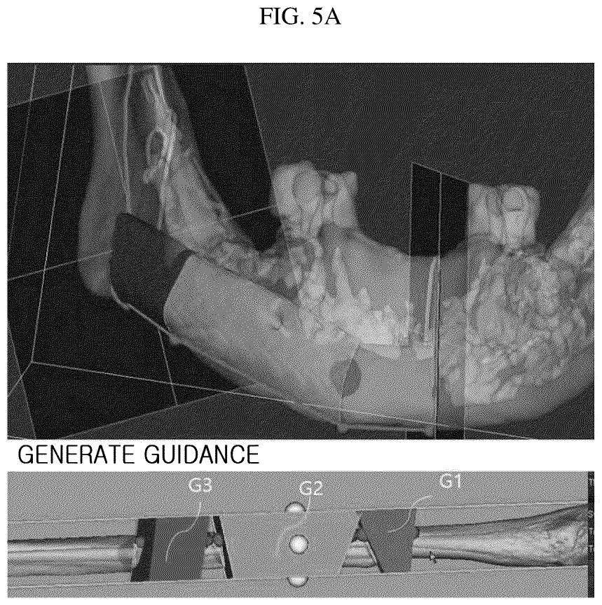

Hereinafter, FIGS. 4A to 4H illustrate a user modeling process of modeling a plurality of cutting target skeletal images suitable for a plurality of implantation target skeletal images, according to an embodiment of the present invention. The user modeling process described below may be arbitrarily adjusted by the user through the input/output interface 101 of the design apparatus.

FIG. 4A is a screen obtained as a user turns the cutting target image of the fibula such that the three implants displayed in the implantation target image area and the three implants displayed in the cutting target image area simultaneously in FIG. 3E are aligned and displayed.

That is, in the cutting target area on the lower part of FIG. 3E, the first implant, the second implant, and the third implant are displayed in different colors from the left.

In this regard, referring to FIG. 4A, the user may turn the fibula such that the third implant, the second implant, and the first implant are displayed from the left. Accordingly, by vertically comparing with the implantation target area image on the upper part of FIG. 4A, the position of the vertex in the cutting target area may be adjusted, or the position of each implant may be moved upward and downward directions, turned, and spined to better fit into the lower jaw, as will be described in FIGS. 4B to 4G.

First, FIG. 4B illustrates a first adjustment screen obtained by a user arbitrarily adjusting the implantation target area displayed with the three implants overlaid thereon. Specifically, in FIG. 4B, it can be seen that the three implants have been moved upward with respect to the implantation target area image. The generated implant bridge is located upward from the position of the first to fourth vertices extracted initially. This may be used, for example, as an additional adjustment operation in which a surgeon to perform reconstruction using the corresponding low jaw implant checks the skeletal structure of the law jaw in three dimensions and more appropriately fits the implant into the lower jaw.

Next, FIG. 4C illustrates a second adjustment screen obtained by the user arbitrarily adjusting the implantation target area displayed the three implants overlaid thereon. Specifically, FIG. 4C is a screen obtained by turning the three implants with respect to the implantation target area image. It can be seen that the bone in the cutting target area on the lower part of FIG. 4C is also turned synchronously with the turning of the implantation target area. This may be used, for example, as an additional adjustment operation in which a surgeon to perform reconstruction using the corresponding low jaw implant checks the skeletal structure of the law jaw in three dimensions and more appropriately fits the implant into the lower jaw.

Next, FIG. 4D illustrates a third adjustment screen obtained by the user arbitrarily adjusting the implantation target area displayed with three implants overlaid thereon. Specifically, FIG. 4D illustrates a screen obtained by spinning the three implants with respect to the implantation target area image. Specifically, it can be seen that the set of implants turned by the user in FIG. 4C has been spun.

In addition, FIG. 4E illustrates a fourth adjustment screen obtained by a user arbitrarily adjusting the implantation target area displayed with the three implants overlaid thereon. Specifically, in FIG. 4E, it can be seen that the three implants have been moved downward with respect to the implantation target area image. The generated implant bridge is located downward from the position of the first to fourth vertices extracted initially. This may be used, for example, as an additional adjustment operation in which a surgeon to perform reconstruction using the corresponding low jaw implant checks the skeletal structure of the law jaw in three dimensions and more appropriately fits the implant into the lower jaw.

Additionally, referring to FIG. 4F, the user may also adjust the position of the fourth vertex pt4 located on the cutting plane of the implantation target image area. That is, the user may adjust the fourth vertex pt4 in FIG. 4E such that the position of the fourth vertex pt4 is located to the outermost point in which the cutting target bone meets the cutting plane 33, and accordingly, the design apparatus may generate a different guidance based on the changed position of the four vertice pt4.

In the description above, a method for a user to adjust the position of an implant in the implantation target area has been described with reference to FIGS. 4A to 4F.

Hereinafter, a method for a user to adjust the position of an implant in the cutting target area image is described with reference to FIGS. 4G and 4H.

For example, referring to FIG. 4G, the user may also adjust the positions of vertices located on the cutting plane on the cutting target image area. That is, as shown in FIGS. 4G and 4H, it can be seen that the position of each vertex has been moved to secure space between each implant. This may be used, for example, as an additional adjustment operation in which a surgeon to perform reconstruction using the corresponding low jaw implant checks the shape of the fibula in three dimensions and more appropriately locates each vertex to generate a guidance for easy cutting.

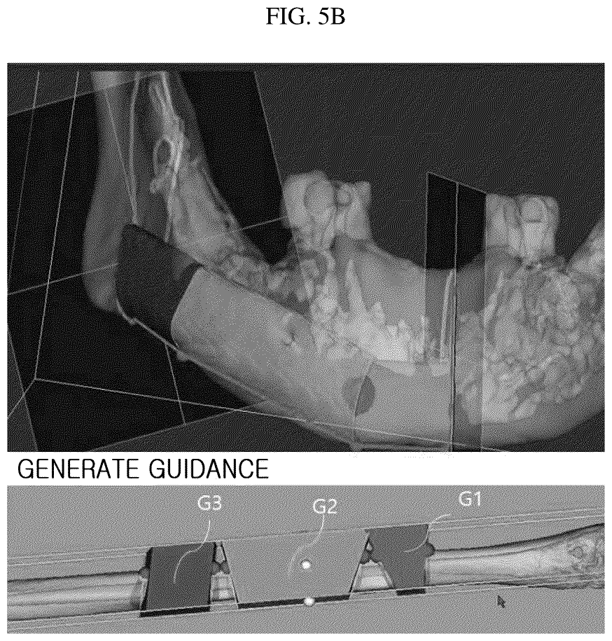

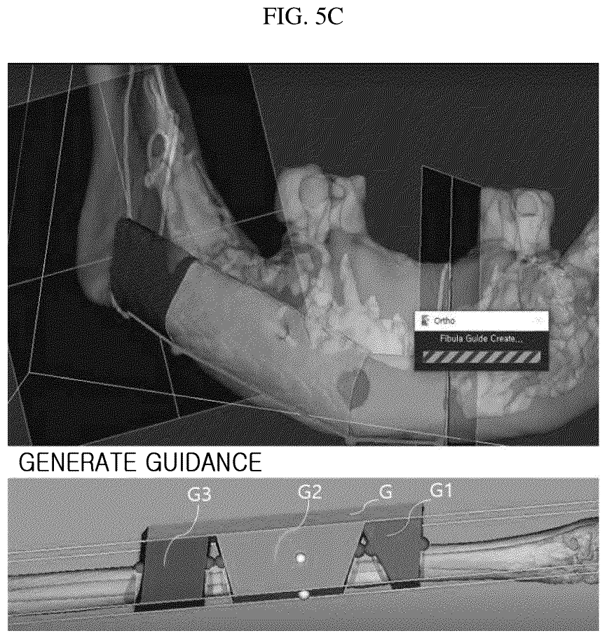

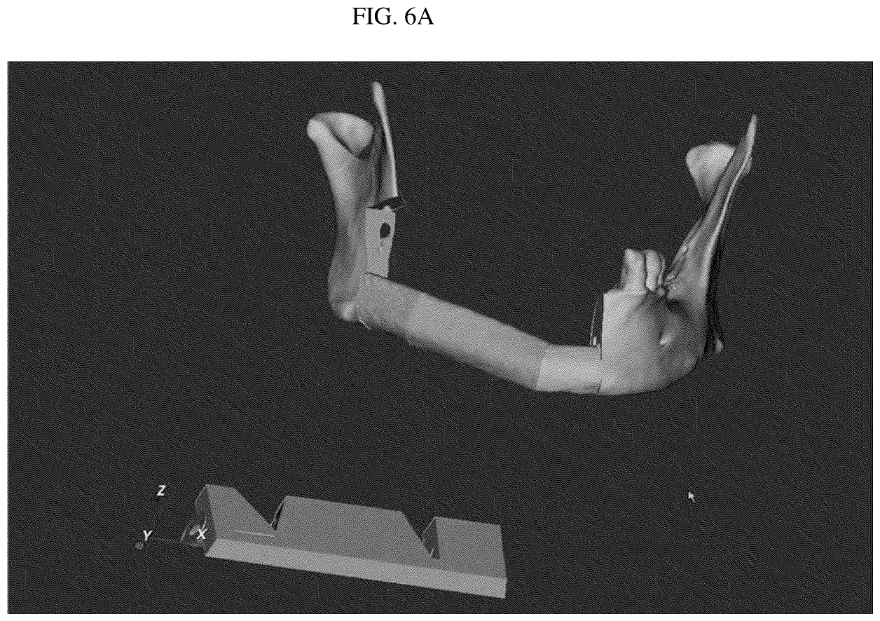

Next, FIGS. 5A to 5C illustrate a process of designing the first guidance according to ab embodiment.

FIG. 5A illustrates guidances generated for a plurality of divided implants. The number of generated guidances may be generated as many as the number of implants generated. For convenience, the guidances generated in FIG. 5A are referred to as first to third guidances G1 to G3.

The design apparatus according to the present invention may display width, depth, and height information about the generated guidance such that the user arbitrarily adjusts the width, depth, and height of the generated guidance.

For example, in FIG. 5B, the height of each guidance is reduced from 20.69 mm to 7.41 mm by the user.

In addition, the design apparatus according to the present invention may display a thickness to be settable by a user such that the plurality of generated guidances G1 to G3 may be designed as one integrated guidance using 3D printing technology.

For example, in FIG. 5C, it can be seen that the first to third guidances have been changed to have a fixing part including a thickness.

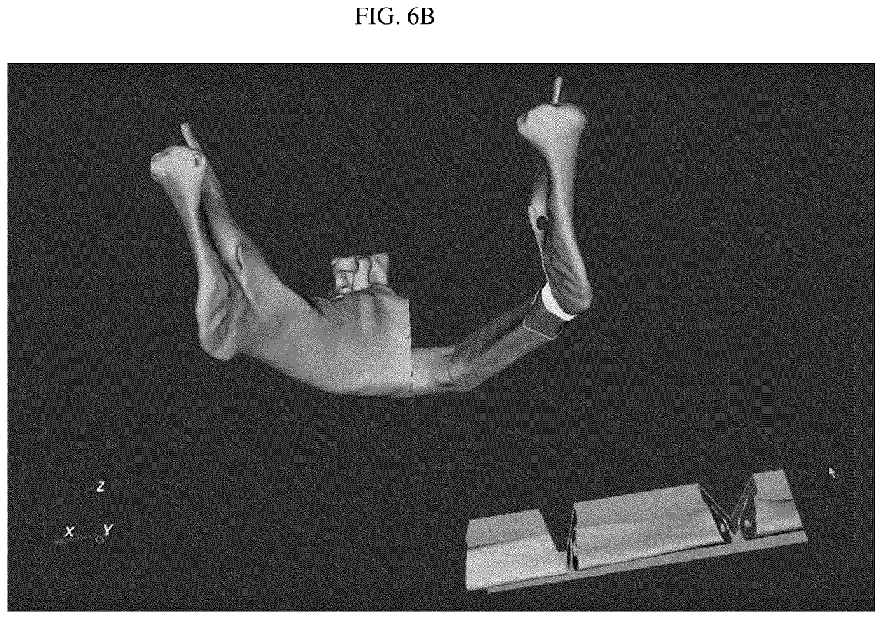

Hereinafter, FIGS. 6A to 6B illustrate a plurality of cutting target skeletal images, which are designed by the first guidance, overlaid on a second implantation target skeletal image, according to an embodiment, which are shown based on the front and rear views.

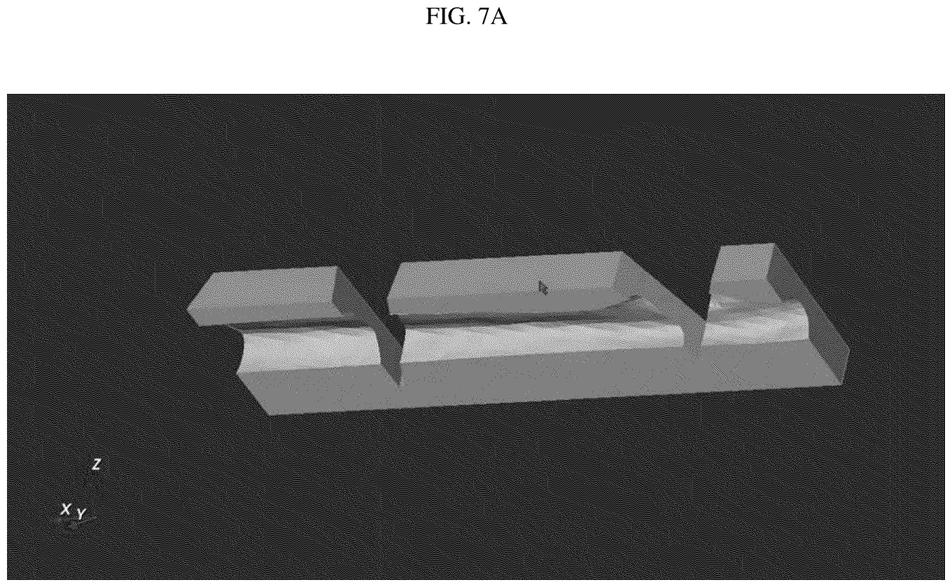

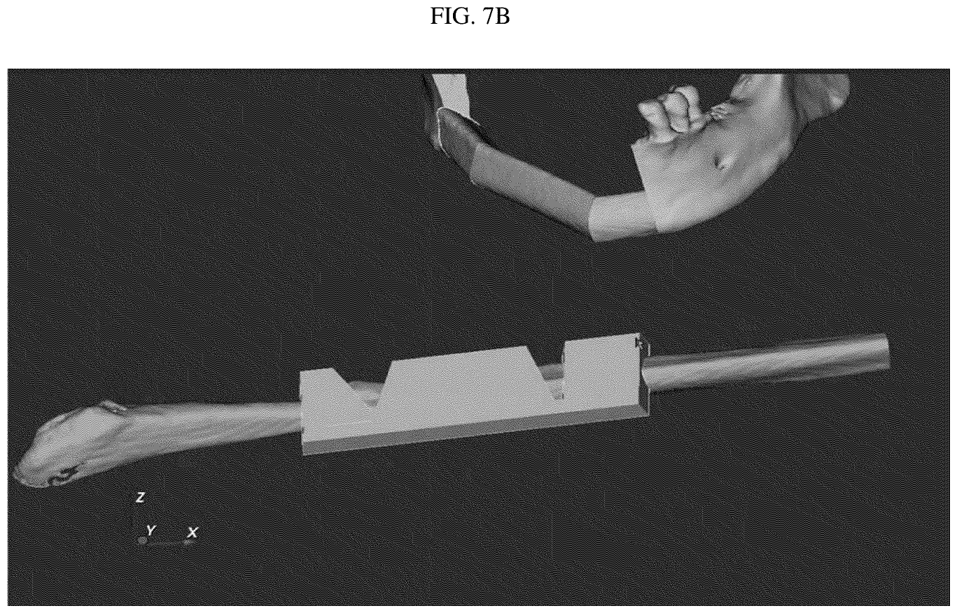

FIG. 7A is a perspective view of the first guidance according to an embodiment, and FIG. 7B is a view of the first guidance overlaid on the cutting target skeletal image, according to an embodiment.

In the description above, a design apparatus that allows a user to perform adjustment has been described. Hereinafter, an embodiment of an algorithm in which a design apparatus automatically perform design from specifying a cutting target area and an implantation target area to generating a guidance without user adjustment is described. In particular, an algorithm for generating a low jaw guidance in implanting the fibula to the low jaw has been described, but the method of designing an implant and a guidance specific to a patient according to the present invention is not limited to the algorithm described below.
Operation 1: a 3D model image of the lower jaw and a 3D model image of the fibula are prepared.
Operation 2: two cutting planes P₁ and Pₙ₋₁ for removing a specific closed area in the lower jaw are generated.
Operation 3: lower jaw lines that pass through the two cutting planes are designated.
Operation 3 includes 1) designating a reference line for disposing a fibula in place of a removed lower jaw area, 2) designating a line along the lower end of the lower jaw, 3) selecting n vertices (vertex: v₁ to vₙ), and 4) generating n−1 lines L₁ to Lₙ₋₁.
Operation 4: cutting planes are generated at points v₂ to vₙ₋₁.

Specifically, in operation 4, as an example, the cutting plane pₓ at the vertex vₓ is generated by using the vertex vₓ as the origin and rotating a plane, which has an outer product of a line Lₓ₋₁ and a line Lₓ as a normal vector, half the angle between the line Lₓ₋₁ and the line Lₓ with respect to the normal vector.
Operation 5: the lower jawlines are aligned in a straight line.
Operation 5 is: 1) a line Lₙ is rotated based on a line Lₙ₋₁ to form a straight line, 2) the cutting planes Pₓ and Pₓ₊₁ adjacent to a line Lₓ are rotated together with the line Lₓ, and 3) the cutting plane Pₓ₊₁ between the cut planes, adjacent to the line Lₓ is duplicated and rotated, and in this case, the duplicated cutting plane is defined as a duplicated cutting plane DPₓ₊₁. 4) the operation is repeated on all lower jaw lines with respect to the cutting planes P₁ to Pₙ₋₁, and 5) the rotation matrix mx and the inverse matrix imx of each operation are stored.
Operation 6: an oriented bounding box (OBB) of a fibula model is generated. Specifically, a fibula line is defined by connecting center points of the two smallest planes of the OBB.
Operation 7: the aligned lower jaw line is moved and rotated to match the fibula line.
1) The cutting planes P₁ to Pₙ and the duplicated cutting planes DP₂ to DPₙ₋₁ are also rotated by as much as the lower jaw line is rotated, 2) a transformation matrix used for the movement and rotation is M1, and 3) an inverse matrix of M is defined as im1.
Operation 8: the fibula is moved and rotated based on the aligned lower jaw line.
1) The distance the fibula moved from the lower jaw line is d, 2) The angle at which the fibula model is rotated based on the fibula line is r1, 3) The angle at which the fibula model is rotated based on the aligned lower jaw line is r2, 4) The transformation matrix of d, r1, and r2 is defined as M2, and 5) the inverse matrix of M2 is defined as im2.
Operation 9: the fibula model is cut with the cutting planes Pₓ and DPₓ₊₁, and the middle piece is defined as FPₓ (FP: Fibula piece).
1) The operation is repeated for the cutting planes P₁ to Pₙ₋₂, and 2) For Pₙ₋₁, cutting is performed with Pₙ₋₁ and Pₙ.
Operation 10: iM2, iM1, and iMx are applied to FPₓ, and the resultant FPₓ is moved to the cutting area of the lower jaw. 1) That is, the operation is repeated for all fibular pieces FPs including Fp₁ to FPₙ₋₁.
Operation 11: operation 9 is repeated based on the OBB of the fibula model to generate cutting guidance pieces GPs including GP₁ to GPₙ₋₁.
Operation 12: a bridge structure connecting all cutting guidance pieces GPs is generated, and all the cutting guidance pieces GPs and the bridge into one part to generate a fibula guidance model.

FIG. 8 is a block diagram illustrating the structure of a design apparatus according to an embodiment.

According to the embodiment, the electronic apparatus 100 may include an input/output interface 101, a memory 103, and a processor 105. In the embodiment, the electronic apparatus 100 may be connected to an external server or database through a transceiver or communication interface, and exchange data.

The processor 105 may perform at least one method described above with reference to FIGS. 1 to 7C. The memory 103 may store information for performing at least one method described above with reference to FIGS. 1 to 7C. The memory 103 may be a volatile memory or a non-volatile memory.

The processor 105 may control the electronic apparatus 100 to execute programs and provide information. Program code executed by the processor 105 may be stored in the memory 103.

The processor 105 may, in connection with the memory 103, specify a first skeletal image of a patient's skeleton in which an affected region is located, extract a plurality of vertices, model a plurality of cutting target areas based on the plurality of vertices in a second skeletal image, receive an input of arbitrary adjustment of the modeled plurality of cutting target areas from a user, generate a guidance suitable for the plurality of cutting targets, and display the plurality of cutting targets to be overlaid on the first skeletal image.

In FIG. 8, the electronic apparatus 100 shown in FIG. 8 includes only components related to the disclosed embodiment. Accordingly, those skilled in the art may understand that other general-purpose components may be included in addition to the components shown in FIG. 8.

The apparatus according to the above embodiments may include a processor, a memory for storing and executing program data, a permanent storage such as a disk drive, and a user interface apparatus, such as a communication port for communicating with an external apparatus, a touch panel, a key, a button, and the like. Methods implemented with software modules or algorithms may be stored on a computer readable recording medium as computer readable codes or program instructions executable on the processor. Here, the computer-readable recording media include a magnetic storage medium (e.g., a read-only memory (ROM), a random-access memory (RAM), a floppy disk, a hard disk, and the like), an optical readable medium (e.g., a compact disk (CD)-ROM, a digital versatile disk (DVD), etc.) and other recording media. The computer-readable recording medium may be distributed over computer systems connected through a network such that computer readable codes may be stored and executed in a distributed manner. The medium may be readable by a computer, stored in a memory, and executable in a processor.

The embodiments may be represented by functional block configurations and various processing operations. These functional blocks may be implemented with any number of hardware and/or software configurations that perform particular functions. For example, the embodiments may adopt integrated circuit configurations such as memory, processing, logic, look-up tables, etc., which may perform various functions by control of one or more microprocessors or by other control apparatuses. Similar to the way in which components may be implemented in software programming or software components, the present embodiments may be implemented in a variety of ways, including C, C++, Java, an assembler, python, and the like. Functional aspects may be implemented with algorithms running on one or more processors. In addition, the present embodiment may employ conventional techniques for electronic environment setting, signal processing, and/or data processing. Terms such as “mechanism”, “element”, “means”, “configuration” may be used broadly and are not limited to mechanical and physical configurations. The term may include the meaning of a series of routines of software in conjunction with a processor or the like.

As is apparent from the above, the method disclosed herein is implemented to reduce the risk in actual bone implantation surgery can by identifying in advance the correct method of cutting the skeleton, the cutting position, and the like.

In addition, the method disclosed herein is implemented to design a guidance that allows a cutting part to be intuitively identified by contacting the patient's skeleton, thereby controlling errors occurring due to environmental differences between the virtual three-dimensional space and the real space.

The effects of the present invention are not limited to those described above, and other effects not described above will be clearly understood by those skilled in the art from the above detailed description.

The above-described embodiments are merely examples and other embodiments may be implemented within the scope of the claims described below.

What is claimed is:

1. A design method, which is a method performed by an apparatus for designing an implant and a guidance specific to a patent, the design method comprising:
specifying a first skeletal image of a skeleton of a patient in which an affected region to which an implant is to be implanted is located;
specifying a second skeletal image of another region skeleton including the implant;
calculating a number of at least one implant matching the first skeletal image;
cutting the second skeletal image according to the calculated number of implants such that the at least one implant matches the first skeletal image, and displaying the cut second skeletal image; and
displaying the cut at least one implant to be overlaid on the first skeletal image.

2. The design method of claim 1, wherein
the specifying of the first skeletal image includes:
generating one or more planes around the affected region; and
specifying, as the first skeletal image, a specific closed area corresponding to a cutting target area among one or more closed areas defined in the first skeletal image by the one or more planes.

3. The design method of claim 1, wherein the calculating of the number of the at least one implants matching the first skeletal image includes
specifying, by a user, coordinates of n vertices inside or outside the first skeletal image.

4. The design method of claim 3, wherein the cutting of the second skeletal image according to the calculated number of implants such that the at least one implant material matches the first skeletal image, and the displaying of the cut second skeletal image includes:
generating two cutting planes to remove the specific closed area;
designating first lines passing through the two cutting planes;
generating cutting planes n−1 from the coordinates of the vertices;
aligning the first lines into a straight line;
generating an oriented bounding box (OBB) for the other area skeleton including the second bone image, and connecting center points of two minimum planes of the OBB to designate a second line;
matching the first line with the second line; and
cutting the other region skeleton based on the first line.

5. The design method of claim 4, further comprising:
generating cutting guide pieces based on the OBB for the second skeletal image;
generating a bridge structure connecting the cutting guide pieces; and
generating a guidance by combining the cutting guide pieces and the bridge structure.

6. The design method of claim 1, wherein the displaying of the cut at least one implant to be overlaid on the first skeletal image includes
when a user arbitrarily moves the coordinates of the vertex, performing the cutting of the second skeletal image according to the calculated number of implants such that the at least one implant matches the first skeletal image and the displaying of the cut second skeletal image again.

7. The design method of claim 1, wherein the displaying of the cut at least one implant to be overlaid on the first skeletal image includes
allowing a user to arbitrarily perform coordinate change, turning, or spinning on the cut at least one implant displayed to be overlaid on the first skeletal image.

8. The design method of claim 5, wherein the displaying of the cut at least one implant to be overlaid on the first skeletal image includes
displaying the at least one implant to be overlaid on the first skeletal image while displaying the at least one implant with respect to the other region skeleton including the second skeletal image.

9. A computer readable recording medium on which a program for performing the method according to claim 1 is recorded.

10. An apparatus for designing an implant and a guidance specific to a patent, the apparatus comprising:
an input/output interface, a memory in which an instruction is stored, and a processor,
wherein the processor is configured to, in connection with the memory,
specify a first skeletal image of a skeleton of a patient in which an affected region to which an implant is to be implanted is located;
specify a second skeletal image of another region skeleton including the implant;
calculate a number of at least one implant matching the first skeletal image;
cut the second skeletal image according to the calculated number of implants such that the at least one implant matches the first skeletal image, and display the cut second skeletal image; and
display the cut at least one implant to be overlaid on the first skeletal image.
